(19) United States
(12) Patent Application Publication
(10) Pub. No.: US 2023/0400360 A1
(43) Pub. Date: Dec. 14, 2023

(54) ELECTRICAL ASSEMBLY WITH MULTI-ZONE TEMPERATURE MONITORING

(71) Applicant: GENTHERM MEDICAL LLC, Cincinnati, OH (US)

(72) Inventor: Rüdiger SPILLNER, Augsburg (DE)

(21) Appl. No.: 18/033,917

(22) PCT Filed: Dec. 7, 2021
(86) PCT No.: PCT/IB2021/000859

§ 371 Date: Apr. 26, 2023

(30) Foreign Application Priority Data
Dec. 31, 2020 (DE) 10 2020 007 924.5

(51) Int. Cl. G01K 7/16; G01K 1/02; G01K 3/00; H05B 3/34; A61F 7/00

(52) U.S. Cl. CPC G01K 7/16; G01K 1/026; G01K 3/005; H05B 3/342; A61F 7/007; A61F 2007/0096

(57) ABSTRACT

The invention relates to an electrical assembly (10) having multi-zone temperature monitoring, comprising: a heat-generating electrical device (12); a measurement circuit (18) having a plurality of temperature-dependent electrical shunt resistors (20, 20′, 20″, 20a-20h), wherein the shunt resistors (20, 20′, 20″, 20a-20h) are positioned in mutually spaced temperature-measurement regions (22a-22h) of the heat-generating electrical device (12); and an analysis device (28) which has a measurement channel (26) for detecting measurement values, wherein the shunt resistors (20, 20′, 20″, 20a-20h) are electrically conductively connected to the measurement channel (26) of the analysis device (28) via a common measurement line (24), and the analysis device (28) is designed to determine a temperature and/or a temperature limit value being exceeded in at least one of the temperature-measurement regions (22a-22h) by analyzing the signal at the measurement channel (26).

The invention relates to an electrical assembly having multi-zone temperature monitoring, comprising: a heat-generating electrical device, a measurement circuit having a plurality of temperature-dependent electrical measuring resistors, wherein the measuring resistors are positioned in mutually spaced-apart temperature-measurement regions of the heat-generating device, and an analysis device that has a measurement channel for detecting measured values.

The invention further relates to a heating device with an electrical assembly having multi-zone temperature monitoring.

Moreover, the invention relates to a cell connector with an electrical assembly having multi-zone temperature monitoring.

Known electrical assemblies having multi-zone temperature monitoring use a plurality of components, such as, for example, heat or cold conductors or other thermoelements, for measuring temperature. In the past, each thermoelectrical component has required discrete wires, cables, and connectors. Furthermore, the analysis device used has required a plurality of measurement channels via which the signals from the thermoelectrical components can be analyzed for measuring temperature. Overall this leads to relatively high usage of materials and the need for a large amount of space in terms of packing and installation space. Moreover, the known electrical assemblies of the multi-zone temperature monitoring require relatively complex assembly.

The underlying object of the invention is thus to simplify multi-zone temperature monitoring of electrical components.

The object is attained using an electrical assembly of the aforesaid type, wherein the measuring resistors of the inventive electrical assembly are electrically conductively connected to the measurement channel of the analysis device via a common measurement line and the analysis device is designed to determine a temperature and/or a temperature limit value being exceeded in at least one of the temperature-measurement regions by analyzing the signal on the measurement channel.

The inventive electrical assembly makes it possible to regulate or limit temperature, wherein only one measurement line is used despite the monitoring of a plurality of temperature-measurement regions. Thus, despite the only one measurement line, it is possible to monitor a large surface-area monitoring region by means of a plurality of measuring resistors. Thus, with a comparatively simple electrical assembly it is possible to realize multi-zone temperature monitoring by means of which local increases in temperature, so-called hot-spots, can be reliably detected on heat-generating electrical devices.

The electrical assembly or its measurement circuit can be realized, for example, as surface-mounted components (SMD components). A comparatively low requirement for materials results overall. With respect to packing and installation space, the electrical assembly furthermore requires a relatively small amount of space. In addition, assembling the electrical assembly is relatively simple, since it is possible to do without separate cabling and separate measurement channels for each of the electrical measuring resistors.

The electrical assembly can be employed, for example, in connection with disposable articles, such as, for example, patient warmers. The heat-generating electrical device may be, for example, a warming mat or warming blanket in which the measurement circuit is integrated. The analysis device can be a component of a control and/or regulation device. The control and/or regulation device including the analysis device can be provided for multiple uses, for example, and can be connectable to different warming mats and/or warming blankets. In general, the electrical assembly offers advantages wherever there are stringent integration or packing requirements and/or a large number of similar sensors are required. Thus, the inventive electrical assembly can also be used in connection with cell terminal boards or cell sensor boards.

In one preferred embodiment of the inventive electrical assembly, the measured value detectable by the analysis device is the current flowing through the measurement channel and/or the voltage applied to the measurement channel. In this way, the analysis device is designed to determine a temperature and/or a temperature limit being exceeded in at least one of the temperature-measurement regions using an analysis of the current flowing through the measurement channel and/or the voltage applied to the measurement channel.

In one further preferred embodiment of the inventive electrical assembly, the electrical resistance value of the measuring resistors changes, at least in one temperature range, non-linearly with respect to a temperature change in the specific temperature-measurement regions. In the region of a temperature limit, the measuring resistors have a conductivity anomaly that leads to a sharp change in the conductivity of the measuring resistors in this temperature region. If the measuring resistors heat up, the electrical resistance value of the measuring resistors can decrease sharply if the temperature limit is exceeded. When the measuring resistors cool down, the electrical resistance value of the measuring resistors can increase sharply if the temperature limit value is not met.

In another preferred embodiment of the inventive electrical assembly, the measuring resistors are embodied, at least in part, from vanadium dioxide. If vanadium exceeds a temperature limit of approx. 68 degrees Celsius, the crystal structure of the vanadium dioxide changes. There is a rutile crystal structure in the metallic phase for temperatures above the temperature limit of 68 degrees Celsius, while the insulating or semiconducting phase has a monocline structure below the temperature limit of 68 degrees Celsius. The electrical conductivity of vanadium dioxide changes by the factor 10³ to 10⁵ during phase transition.

In one further preferred embodiment of the inventive electrical assembly, the measuring resistors and/or the measurement line are integrated into the heat-generating electrical device. The measuring resistors and/or the measurement line are preferably electrically conductively connected to a voltage and/or current source of the heat-generating electrical device. The heat-generating electrical device can be, for example, a heating device having a plurality of heat conductors or a plurality of heat conductor segments. The heat conductors or heat conductor segments can be tracks on a heating film. The measuring resistors are preferably electrically conductively connected to the heat conductors or heat conductor segments. The measuring resistors are preferably switched in parallel to the heat conductors or heat conductor segments.

In one refinement of the inventive electrical assembly, the analysis device is designed to determine the temperature or a temperature limit being exceeded in at least one of the temperature-measurement regions by detecting a measurement threshold being exceeded or not reached on the measurement channel. If there is an elevated temperature in at least one of the temperature-measurement regions, the resistance value of the measuring resistor arranged in this temperature-measurement region decreases. A higher current will flow from the heat-generating electrical device via the measuring resistors to the measurement channel or the potential will change correspondingly. The analysis device can monitor a current limit being exceeded, so that then the temperature limit is also detected.

Moreover, an inventive electrical assembly is advantageous in which the measurement circuit is designed such that the measurement threshold on the measurement channel is higher than the maximum measured value on the measurement channel that can be caused by an increase in temperature below the temperature limit in all temperature-measurement regions.

Alternatively, the measurement circuit is designed such that the measurement threshold on the measurement channel is lower than the minimum measured value on the measurement channel that can be caused by an increase in temperature below the temperature limit in all temperature-measurement regions. Thus the analysis device is able to distinguish between a large change in the measurement value on the measurement channel due to a temperature limit being exceeded in one measurement region, on the one hand, and a large change in the measurement value due to a flat increase in temperature in a plurality of temperature-measurement regions below the temperature limit.

In one refinement of the inventive electrical assembly, the analysis device is designed to determine the temperature limit being exceeded in at least one of the temperature-measurement regions by detecting a specific temporal change of the measurement value on the measurement channel. In this case, the interference effects due to the other measuring resistors in the temperature-measurement regions of which the temperature limit being exceeded has not been detected can also be greater than the measurement change resulting from the temperature limit being exceeded in a temperature-measurement region, since now a temporal component is taken into account.

In one further preferred embodiment of the inventive electrical assembly, the analysis device is designed, using the amount of the measurement change on the measurement channel, to identify the temperature-measurement region in which a temperature limit being exceeded has occurred. The measuring resistors are connected to electrical conductors of different length of the heat-generating electrical device. The electrical conductors of the heat-generating electrical device can be heat conductors or heat conductor segments, for example. As the length of the electrical conductors increases, their electrical resistance increases. Thus the current rises and voltage drops across the different measuring resistors differ in magnitude depending on where these resistors are located and the length of the electrical conductor to which they are connected. This effect occurs because the current to the different measuring resistors must flow through segments of different length before it can branch off before the respective measuring resistor.

In one further preferred embodiment of the inventive electrical assembly, the heat-generating electrical device and the measurement circuit are connected to the same ground. Alternatively, the heat-generating electrical device and the measurement circuit are connected to different grounds. Both variants can offer technical advantages, so that a suitable ground connection should be selected depending on the purpose for which the electrical assembly will be used.

In one further preferred embodiment of the inventive electrical assembly, the leads for the respective measuring resistors have different capacitive properties and/or different inductive properties. The analysis device is preferably designed, based on the different capacitive properties and/or the different inductive properties of the leads of the respective measuring resistors, to associate at least one determined temperature and/or at least one determined temperature limit being exceeded with one measuring resistor. The individual properties of the leads change their frequency-dependent complex resistance, that is, their impedance, and also the oscillation behavior (RC, RCL). In this case, the specific measuring resistance detects the temperature, wherein the analysis device can detect, using the different capacitive properties and/or the different inductive properties of the leads, which measuring resistance the measurement change on the measurement channel derives from, so that the associated temperature-measurement region can be determined. In this case, the measuring resistors can also be heat conductors or cold conductors, the electrical resistance of which changes linearly relative to a temperature change in the respective temperature-measurement regions.

In one preferred embodiment of the inventive electrical assembly, the analysis device has integrated circuitry that is designed to check the frequency-dependent total resistance, the oscillation behavior, and/or the pulse response on the measurement channel. The analysis device is preferably designed to check only a few specified frequency ranges. Thus the costs for the circuit technology are kept low. The integrated circuitry can be designed to perform an impedance spectroscopy. The integrated circuitry can take the measurement on a parallel measurement line. Alternatively, for the measurement, the potential of the heat-generating electrical device can be switched, isolated, or brought to a defined level. That is, there is a brief separation of a higher power from the measurement circuit.

In one refinement of the inventive electrical assembly, the capacitive properties of the respective lead are caused by capacitive components in the lead or by the structure of the conductor material of the respective lead. Alternatively or in addition, the inductive properties of the respective lead are caused by inductive components in the lead or by the structure of the lead material of the respective lead. For providing capacitive properties, the structure of the conductor material can be a film with conductive tracks, wherein the tracks engage in one another parallel and thus form numerous small capacitors. The advantage of such an arrangement is that it is easy to produce in the surface. The structure of the conductor material can also include a turning down or fold in which two electrically conductive surfaces, between which there is an insulating intermediate layer or a dielectric medium, are arranged spaced apart from one another such that they form a type of plate capacitor. To a certain degree, the base capacity and the resistance of a resonant circuit can be adjusted by the design of a film circuit, for instance the length of the circle or the length and/or distance of parallel circuits. If a second film or flaps with half-cuts are used, a second layer can be applied to the base film to generate a second capacitor plate. Further capacitor plates can be produced by applying further layers.

Moreover, an inventive electrical assembly is advantageous in which the leads of the respective measuring resistors have different temperature-dependent capacities and/or different temperature-dependent inductances. In this way it is possible for the analysis device to associate a determined temperature or a determined temperature limit being exceeded with a temperature-measurement region. Preferably there is no overlapping of temperature-induced impedance changes/adjustments in the measurement line.

In one further preferred embodiment of the inventive electrical assembly, the heat-generating electrical device is a warming blanket or warming mat having a plurality of heat conductors or heat conductor segments. The heat conductors or heat conductor segments can be conducting tracks of the warming blanket or warming mat. The conducting tracks can be applied to a support layer of the warming blanket or warming mat.

The underlying object of the invention is further attained by a heating device of the type cited in the foregoing, wherein the electrical assembly of the inventive heating device is embodied according to one of the preceding embodiments. Refer to the advantages and modifications of the electrical assembly for advantages and modifications of the inventive heating device.

The heat-generating device of the heating device is preferably a warming blanket or warming mat. The warming blanket can be a warming blanket for people that can be used for covering a person from above or as a base layer for warming a person from below. Such warming blankets are used on operating tables, for example. Furthermore, such warming blankets are also used during rescue operations in the outdoors to protect unconscious or injured persons from hypothermia. Such warming blankets can also be used as a sterile separating layer during rescue operations to protect persons from contact with contaminated soil. The warming blanket of the heating device can be a heating pad. In this case the warming blanket is designed, for example, as an inexpensive disposable product in order to provide sterile surroundings and to prevent infection due to multiple uses.

The analysis device is preferably a component of a control and/or regulating device by means of which multi-zone temperature monitoring and/or heat output monitoring as well as heat output control and/or heat output regulation can be implemented. The control and/or regulating device is generally a more expensive electronic product, so that the control and/or regulating device for multiple use can be separated from the heat-generating device, in particular from the disposable warming blanket.

The underlying object of the invention is furthermore attained by a cell connector of the type cited in the foregoing, wherein the electrical assembly of the inventive cell connector is embodied according to one of the embodiments described in the foregoing. Therefore refer to the advantages and modifications of the inventive electrical assembly for advantages and modifications of the inventive cell connector.

Preferred embodiments of the invention are explained and described in greater detail in the following, referring to the enclosed drawings.

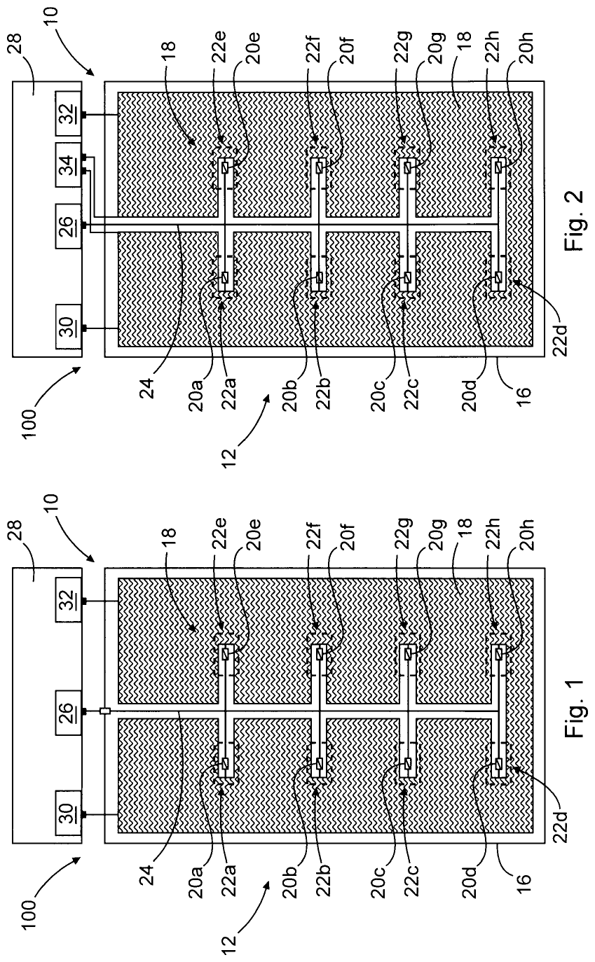

FIG. 1 is a schematic depiction of an exemplary embodiment of the inventive heating device;

FIG. 2 is a schematic depiction of a further exemplary embodiment of the inventive heating device;

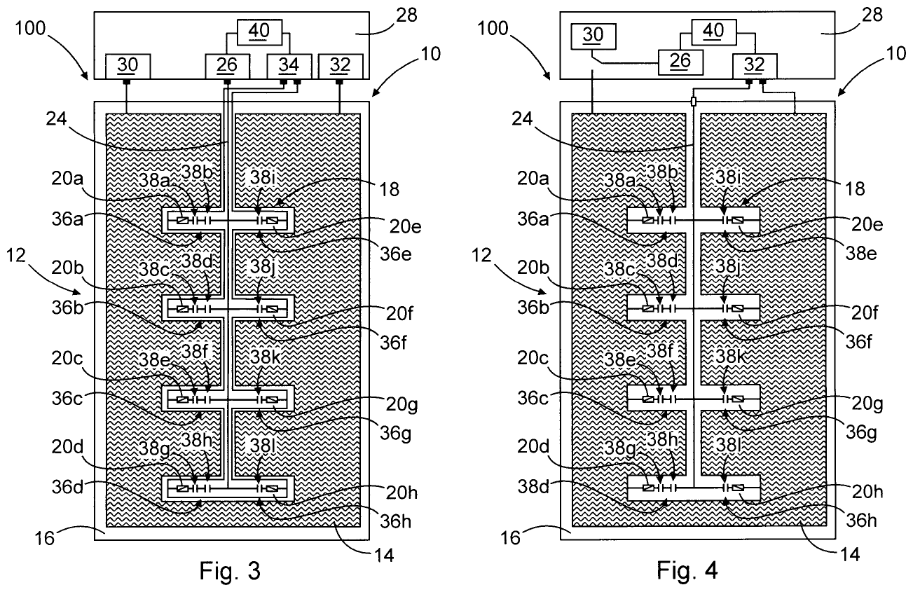

FIG. 3 is a schematic depiction of a further exemplary embodiment of the inventive heating device;

FIG. 4 is a schematic depiction of a further exemplary embodiment of the inventive heating device;

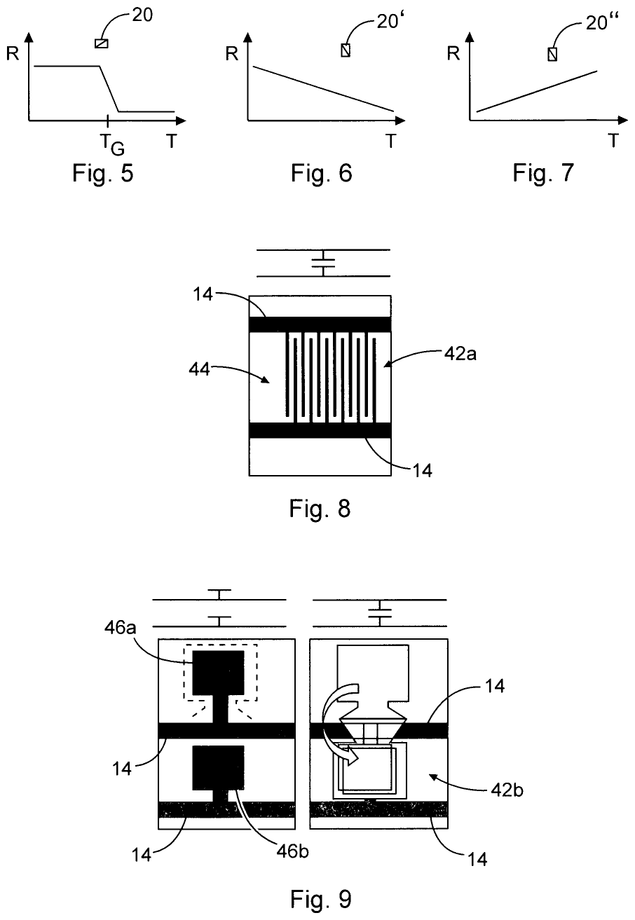

FIG. 5 depicts non-linear temperature-dependent conductivity behavior of a resistor;

FIG. 6 depicts linear temperature-dependent conductivity behavior of a resistor;

FIG. 7 depicts further linear temperature-dependent conductivity behavior of a resistor;

FIG. 8 depicts a conductor tracks structure of a heat-generating electrical device of an inventive assembly; and,

FIG. 9 depicts a fold-over in the conductor tracks structure of a heat-generating electrical device of an inventive assembly.

FIGS. 1 through 4 illustrate different heating devices 100, each with an electronic assembly 10 having multi-temperature monitoring.

The electrical assembly 10 comprises a heat-generating electrical device 12 embodied as a warming blanket. The heat-generating electrical device 12 embodied as a warming blanket comprises a plurality of electrical conductors 14 applied to a support layer 16. The electrical conductors 14 are heat conductors that heat up when supplied with current.

Moreover, the assembly 10 comprises a measurement circuit 18 integrated into the warming blanket 12. The measurement circuit 18 comprises a plurality of temperature-dependent electrical measuring resistors 20a-20h, wherein the measuring resistors 20a-20h are positioned in mutually spaced-apart temperature-measurement regions 22a-22h of the heating device 12. The electrical resistance value R of the measuring resistors 22a-22h in a temperature region changes in a non-linear manner relative to the temperature change in the respective temperature measuring region 22a-22h. The measuring resistors 22a-22h are embodied, at least in part, from vanadium dioxide.

The measuring resistors 22a-22h are connected to a measurement channel 26 of an analysis device 28 via a common measurement line 24.

In the embodiment depicted in FIG. 1, the measuring resistors 22a-22h and the measurement line 24 are electrically conductively connected to a power source 30 to which the heat conductors 14 of the warming blanket 12 are also connected. The measuring resistors 22a-22h are switched in parallel to the heat conductors 14. The warming blanket 12 and the measurement circuit 18 are connected to the same ground 32.

The analysis device 28 is designed to determine a temperature limit being exceeded in one of the temperature measuring regions 22a-22h by analyzing the signal on the measurement channel 26. The measurement value detectable by the analysis device 28 can be the current flowing through the measurement channel 26 and/or the voltage applied to the measurement channel 26.

The crystal structure of the vanadium dioxide changes when the vanadium dioxide of the measuring resistors 20a-20h exceeds the temperature limit T G of approx. 68 degrees Celsius. During the phase transition, the electric conductivity of the vanadium dioxides changes drastically by a factor of 10³ to 10⁵, so that the analysis device 28 can determine the temperature limit being exceeded in a temperature-measurement region 22a-22h by detecting a current threshold being exceeded on the measurement channel 26. When the temperature T in one of the temperature measuring regions 22a-22h is elevated beyond the temperature limit T_{G}, the resistance value R of the measuring resistor 20a-20h arranged in this temperature region 22a-22h decreases sharply. A higher current flows from the heat-generating electrical device 12 via the measuring resistors to the measurement channel 26, so that the analysis device 28 can monitor the current limit being exceeded for detecting a temperature limit being exceeded.

In the exemplary embodiment illustrated in FIG. 2, the heat-generating electrical device 12 and the measurement circuit 18 are connected to different grounds 32, 34. Depending on the application, this can involve technical advantages.

In the exemplary embodiment illustrated in FIG. 3, the leads 36a-36h of the respective measuring resistors 20a-20h have different capacitive properties. The analysis device 28 is designed to associate, based on the different capacitive properties of the leads 36a-36h for the respective measuring resistors 20a-20h, a determined temperature or a determined temperature limit being exceeded with a measuring resistor 20a-20h and thus also with a temperature measuring region 22a-22h. The capacitive properties of the specific lead 36a-36h are brought about by capacitive components 38a-381 in the lead 36a-36h. The individual capacitive properties of the leads 36a-36h each change their frequency-dependent complex resistance and thus also the oscillation behavior. The specific measuring resistor 20a-20h in this case thus detects the temperature T in the temperature-measurement region 22a-22h associated with the specific measuring resistor Using the different capacitive properties of the leads 36a-36h, the analysis device 28 can then determine the measuring resistor 20a-20h from which the change in measured value derives on the measurement channel 26, so that the associated temperature-measurement region 22a-22h can be identified. To this end, the analysis device 28 has integrated circuitry 40 by means of which the frequency-dependent total resistance, oscillation behavior, and pulse response on the measurement channel 26 can be checked.

FIG. 4 illustrates an exemplary embodiment in which the integrated circuitry 40 performs the measurement via a switchable measurement channel 26.

FIG. 5 illustrates the relationship between the resistance value R of a vanadium dioxide measuring resistor 20 and the temperature T. A sharp change in conductivity occurs at around a temperature limit T G. The temperature limit T G for vanadium dioxide resistors 20 is approx. 68 degree Celsius.

At this temperature, the crystal structure undergoes a phase transition from a monocline to a rutile structure. Corresponding vanadium dioxide measuring resistors 20 having a conductivity anomaly at around 68 degrees Celsius can be employed, for example, in the measurement circuits 18 for the embodiment illustrated in FIGS. 1 through 4.

FIGS. 6 and 7 compare the conductivity behavior of measuring resistors 20′, 20″, wherein the characterizing lines of these measuring resistors 20′, 20″ do not undergo a sharp change in conductivity. FIG. 6 relates to a heat conductor that conducts better at high temperatures than at low temperatures. FIG. 7 relates to a cold conductor that conducts better at low temperatures than at high temperatures.

FIG. 8 illustrates by way of example one possibility for adding capacitive properties using a structure 42a of the conductor material. In this case, conductive tracks of a warming mat form the conductor material, wherein a plurality of conductive tracks 44 engage in one another in parallel, so that a plurality of small capacitors are produced by the conductor structure 42a. Such a structure 42a can be employed, for example, in a lead 36a-36h for a measuring resistor 20a-20h (see FIG. 4).

FIG. 9 illustrates production of a conductor track structure 42b, which provides a plate capacitor by turning down or folding electrically conductive surfaces 46a, 46b. Insulating intermediate material or a dielectric medium is disposed between the surfaces 46a, 46b. After being turned down or folded, the surfaces 46a, 46b are spaced apart from one another so that there is an air gap between the surfaces 46a, 46b.

### REFERENCE NUMBERS

10 Assembly
12 Heat-generating electrical device
14 Electrical conductor
16 Support layer
18 Measurement circuit
20, 20′, 20″, 20a-20h Measuring resistors
22a-22h Temperature-measurement regions
24 Measurement line
26 Measurement channel
28 Analysis device
30 Power source
32 Ground
34 Ground
36a-36h Leads
38a-38l Capacitive components
40 Integrated circuitry
42a, 42b Conductor structure
44 Conductor tracks
20 46a, 46b Surfaces
100 Heating device
R Resistance value
T Temperature
T_{G} Temperature limit

1. An electrical assembly having multi-zone temperature monitoring, comprising:
a heat-generating electrical device;
a measurement circuit having a plurality of temperature-dependent electrical measuring resistors, wherein the plurality of temperature-dependent electrical measuring resistors are positioned in mutually spaced-apart temperature-measurement regions of the heat-generating device; and,
an analysis device that has a measurement channel for detecting measured values; wherein the plurality of temperature-dependent electrical measuring resistors are electrically conductively connected to the measurement channel of the analysis device via a common measurement line and the analysis device is designed to determine a temperature and/or a temperature limit value being exceeded in at least one of the mutually spaced-apart temperature-measurement regions by analyzing a signal on the measurement channel.

2. The electrical assembly according to claim 1, wherein the measured values detectable by the analysis device is a current flowing through the measurement channel and/or a voltage applied to the measurement channel.

3. The electrical assembly according to claim 2, wherein an electrical resistance value of the plurality of temperature-dependent electrical measuring resistors changes, at least in one temperature range, non-linearly with respect to a temperature change in the mutually spaced-apart temperature-measurement regions.

4. The electrical assembly according to claim 3,
wherein the plurality of temperature-dependent electrical measuring resistors are fabricated, at least in part, from vanadium dioxide.

5. The electrical assembly according to claim 4,
wherein the plurality of temperature-dependent electrical measuring resistors and/or the common measurement line are integrated into the heat-generating electrical device, wherein the plurality of temperature-dependent electrical measuring resistors and/or the common measurement line are preferably electrically conductively connected to a voltage and/or current source of the heat-generating electrical device.

6. The electrical assembly according to claim 5,
wherein the analysis device is designed to determine the temperature or the temperature limit being exceeded in at least one of the mutually spaced-apart temperature-measurement regions by detecting a measurement threshold being exceeded or not reached on the measurement channel.

7. The electrical assembly according to claim 6, wherein the measurement circuit is designed such that:
the measurement threshold on the measurement channel is higher than a maximum measured value on the measurement channel that can be caused by an increase in temperature below the temperature limit in all of the mutually spaced-apart temperature-measurement regions, or,
the measurement threshold on the measurement channel is lower than a minimum measured value on the measurement channel that can be caused by an increase in temperature below the temperature limit in all of the mutually spaced-apart temperature-measurement regions.

8. The electrical assembly according to claim 7,
wherein the analysis device is designed to determine the temperature limit being exceeded in at least one of the mutually spaced-apart temperature-measurement regions by detecting a specific temporal change of the measurement value on the measurement channel.

9. The electrical assembly according to claim 8,
wherein the analysis device, using an amount of a measurement change on the measurement channel, identifies the mutually spaced-apart temperature-measurement regions in which the temperature limit being exceeded has occurred.

10. The electrical assembly according to claim 9,
wherein the heat-generating electrical device and the measurement circuit are connected to a ground that is the same or grounds that are different.

11. The electrical assembly according to claim 1,
wherein leads for the plurality of temperature-dependent electrical measuring resistors, respectively, have different capacitive properties and/or different inductive properties, wherein the analysis device, based on the different capacitive properties and/or the different inductive properties of the leads, associates at least one determined temperature and/or at least one determined temperature limit being exceeded with one of the plurality of temperature-dependent electrical measuring resistor.

12. The electrical assembly according to claim 11,
wherein the analysis device has integrated circuitry that checks a frequency-dependent total resistance, oscillation behavior, and/or a pulse response on the measurement channel.

13. The electrical assembly according to claim 12, wherein
the capacitive properties of the lead are caused by capacitive components in the leads or by a structure of conductor material of the leads, and/or
the inductive properties of the leads are caused by inductive components in the leads or by a structure of lead material of the leads.

14. The electrical assembly according to claim 13,
wherein the leads of the plurality of temperature-dependent electrical measuring resistors, respectively, have different temperature-dependent capacities and/or different temperature-dependent inductances.

15. The electrical assembly according to claim 10,
wherein the heat-generating electrical device is a warming blanket or a warming mat having a plurality of heat conductors or heat conductor segments.

16. A heating device comprising
an electrical assembly having multi-zone temperature monitoring according to claim 1.

17. A cell connector comprising
an electrical assembly having multi-zone temperature monitoring according to claim 1.
